(19) United States
(12) Patent Application Publication
(10) Pub. No.: US 2024/0277229 A1
(43) Pub. Date: Aug. 22, 2024

(54) INTRAORAL SCANNER USING COMMON-PATH OPTICAL COHERENCE TOMOGRAPHY

(71) Applicant: Dental Imaging Technologies Corporation, Quakertown, PA (US)

(72) Inventors: Siyu MA, East Rochester, NY (US); Xiaodong TAO, East Rochester, NY (US); Victor C. WONG, Pittsford, NY (US)

(21) Appl. No.: 18/573,848

(22) PCT Filed: Jun. 30, 2022
(86) PCT No.: PCT/US2022/035778

§ 371 Date: Dec. 22, 2023

Related U.S. Application Data
(60) Provisional application No. 63/216,901, filed on Jun. 30, 2021.

(51) Int. Cl. A61B 5/00

(52) U.S. Cl. CPC A61B 5/0066; A61B 5/0088; A61B 2562/0233

(57) ABSTRACT

An optical coherence tomography scanner for imaging an intraoral sample has a wavelength-tunable light source configured to generate scanning light having a range of wavelengths and a scanning probe having a scanning head. A light circulator is configured to direct the scanning light to a first sample arm having at least a first optical fiber for conveying light to the sample, to direct a sample signal, having scattered and reflected light from the sample, back from the first optical fiber, to a detector, and to direct a reference signal, having light reflected back along the first optical fiber from a partially reflective surface at the scanning head, to the detector. The detector forms a digital output signal indicative of interference of the combined sample and reference signals. A display is configured to form an image of sample features according to the digital output signal.

### CROSS-REFERENCE TO RELATED APPLICATIONS

This patent application claims priority from U.S. Provisional Appl. Ser. No. 63/216,901, filed Jun. 30, 2021, which is incorporated herein by reference in its entirety.

### TECHNICAL FIELD

The disclosure relates generally to hand-held intraoral optical coherence tomography (OCT) imaging and, more particularly, to apparatuses and methods for scanning using common-path OCT principles.

### BACKGROUND

Optical coherence tomography (OCT) is a non-invasive imaging technique that employs interferometric principles to obtain high resolution, cross-sectional tomographic images that characterize the depth structure of a sample. Particularly suitable for in vivo imaging of human tissue, OCT has shown its usefulness in a range of biomedical research and medical imaging applications, such as in ophthalmology, dermatology, oncology, and other fields, as well as in ear-nose-throat (ENT) and dental imaging.

OCT has been described as a type of “optical ultrasound”, imaging reflected energy from within living tissue to obtain cross-sectional data. In an OCT imaging system, light from a wide-bandwidth source, such as a super luminescent diode (SLD) or other light source, is directed along two different optical paths: a reference arm or path of known optical path length and a sample arm or path that illuminates the tissue or other subject under study. Reflected and back-scattered light from the reference and sample arms is then recombined in the OCT apparatus and interference effects are used to determine characteristics of the surface and near-surface underlying structure of the sample. Interference data can be acquired by rapidly scanning the illumination across the sample. At each of several thousand points along the sample surface, the OCT apparatus obtains an interference profile which can be used to reconstruct an A-scan with an axial depth into the material that is largely a factor of light source coherence. For most tissue imaging applications, OCT uses broadband illumination sources and can provide image content at depths of up to a few millimeters (mm).

There are significant limitations to the various techniques and approaches that have been applied to the problems of intraoral imaging. Constraints on camera and scanner size and form factor and the confined space requirements of the intraoral imaging environment make it challenging to accurately characterize intraoral surfaces. It can be difficult to focus with accuracy on individual surface features, to provide image content of broad areas of patient dentition at suitable resolution and focus, and to provide sufficient illumination for diagnostic purposes.

Accurate imaging of the tooth and other intraoral structures can be compromised due to the effects of fluids. Water, saliva, blood, and other fluids that can collect on and around the teeth can cause difficulties for OCT as well as for reflective imaging systems. For some illumination apparatus, only a portion of the projected light impinges onto the tooth surface, sample S. Similarly, the backscattered light from the tooth surface is again refracted at the fluid-air interface and captured by a camera at another angle. Back-ray tracing of the projection beam and captured light beam locates an intersection point which is shifted toward the imaging system, causing image distortion.

In addition to dimensional inaccuracy, reflection from fluids in the mouth can produce shining spots on images, saturated due to high reflection levels. Still other problems that can be particularly pronounced for intraoral imaging include tight space constraints, fogging, wetness from blood/saliva/water, translucency of teeth, high levels of light absorption and scattering by gum/cheek/tongue, and patient gag reflex, for example. For reasons such as these, intraoral imaging presents considerable challenges over and above problems encountered with most other biomedical imaging applications, with respect to operation, environment, and image quality.

Conventional OCT systems adapt the architecture of an interferometer, which typically consists of both fiber-based and free-space optics and mechanical components, such as one or more fiber couplers, fiber circulators, lenses, and mirrors, for directing light to and from its sampling and reference arms. Acquiring reliable and accurate interference signals requires precision matching of the sampling and reference arm optical paths. An adjustable mechanical reference arm is often used for achieving the optimal optical path length. This requirement, however, presents some inherent difficulties, including the following:
a. added size, weight, and cost to the scanning apparatus. An adjustable mechanical reference arm often employs multiple optical mounts, translation stages, kinematic mounts, and optics components. To prevent the contamination of mirror or lens elements, the optical system often needs a specially designed enclosure, adding further cost, weight, and bulk to the system and making it poorly adapted to a clinical or dental chair setting.
b. sensitivity to vibration and mechanical drift. Because the reference arm often includes a number of components including a free space fiber coupler, it can be very sensitive to environmental vibration. Temperature change may also cause mechanical drift in the reference arm, potentially compromising image quality.
c. high maintenance and downtime costs. To maintain high fiber coupling efficiency, regular realignment is often required, such as on a yearly or monthly basis. This type of adjustment often needs to be performed by specialists, which increases maintenance cost and downtime cost.
d. increased cost of manufacture. To provide high fiber coupling efficiency, special adjustments need to be performed for tuning the reference arm, extending the time needed for final assembly and test.
e. high insertion loss of the reference arm. The conventional free-space reference arm often has higher insertion loss because of the low coupling efficiency of the free space fiber coupler.

These problems become increasingly more complex for an OCT scanning device that uses multiple scanning channels. Improvements that reduce size and cost and help to eliminate sources of mechanical drift and sensitivity would be beneficial for making OCT imaging more usable, robust, and affordable.

### SUMMARY

An object of the present disclosure is to advance the art of intraoral OCT imaging. An embodiment of the present disclosure particularly addresses the need for improved methods for streamlining the OCT design using common-path optical coherence tomography.

Another object of this application is to address, in whole or in part, at least the foregoing and other deficiencies in the related art.

It is a related object of this application to provide, in whole or in part, at least the advantages described herein.

These objects are given only by way of illustrative example, and such objects may be exemplary of one or more embodiments of the application. Other desirable objectives and advantages inherently achieved by the disclosed apparatuses and methods may occur or become apparent to those skilled in the art. The invention is defined by the appended claims.

According to one aspect of the disclosure, there is provided an optical coherence tomography scanner for imaging an intraoral sample, the scanner comprising:
a wavelength-tunable light source configured to generate scanning light having a range of wavelengths;
a scanning probe having a scanning head that directs light to the sample;
a light circulator configured:
   to direct the scanning light through at least a first optical fiber for conveying light to the scan head;
   to direct a sample signal, having scattered and reflected light from the sample and through the at least the first optical fiber, to a detector;
   to direct a reference signal, having light reflected back from a partial reflection apparatus through the at least the first optical fiber, to the detector;
   wherein the detector forms a digital output signal indicative of interference of the combined sample and reference signals; and
   a display configured to form an image of sample features according to the digital output signal.

### BRIEF DESCRIPTION OF THE DRAWINGS

The foregoing and other objects, features, and advantages of the invention will be apparent from the following more particular description of the embodiments of the disclosure, as illustrated in the accompanying drawings. The elements of the drawings are not necessarily to scale relative to each other.

FIG. 1 is a schematic diagram showing an exemplary swept-source OCT (SS-OCT) apparatus according to an embodiment of the present disclosure.

FIG. 2A shows a schematic representation of scanning operation for obtaining a B-scan.

FIG. 2B shows an OCT scanning pattern for C-scan acquisition.

FIG. 3A is a schematic diagram that shows a high-speed intraoral OCT system of the present disclosure having multiple channels.

FIG. 3B is a schematic diagram that shows components that collimate, focus, and scan light from each channel.

FIG. 3C is a schematic diagram showing a channel with an additional camera for viewing the imaged sample.

FIG. 4A shows a schematic for an apparatus using a one-dimensional array for providing output beams from multiple channels.

FIG. 4B shows a schematic for an apparatus using a two-dimensional array for providing output beams from multiple channels.

FIG. 5A is a schematic showing an arrangement of intraoral probe components for a single channel using a common path for reference and signal arms.

FIG. 5B is a schematic diagram for the intraoral probe including light circulation for multiple channels.

FIG. 5C is a schematic diagram for the intraoral probe using a partially reflective surface at the end of the light guide.

FIG. 5D is a schematic for the intraoral probe using a beam splitter and a mirror as the partial reflection apparatus.

FIG. 5E is a schematic for the intraoral probe using a mirror as the partial reflection apparatus.

FIG. 5F is a schematic for the intraoral probe using two mirrors as the partial reflection apparatus.

FIG. 6 shows various arrangements that can be used for the fiber light guide of FIG. 5.

### DETAILED DESCRIPTION

The following is a detailed description of exemplary embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

The general term “scanner” relates to an optical system that is energizable to project a scanned light beam of light, such as broadband near-IR (BNIR) light that is directed to the tooth surface through a sample arm and acquired, as reflected and scattered light returned in the sample arm, for measuring interference with light from a reference arm used in OCT imaging of a surface. The term “scanner” can also refer to a scanning optical element, such as an actuable MEMS (micro-electromechanical systems) scanner, mirror, or mirror array, for example. The term “raster scanner” relates to the combination of hardware components that sequentially scan light toward uniformly spaced locations along a sample, as described in more detail subsequently.

In the context of the present disclosure, the phrase “imaging range” relates to the effective distance (generally considered in the z-axis or A-scan direction) over which OCT measurement is available. The OCT beam is considered to be within focus over the imaging range. Image depth relates to imaging range, but has additional factors related to signal penetration through the sample tooth or other tissue.

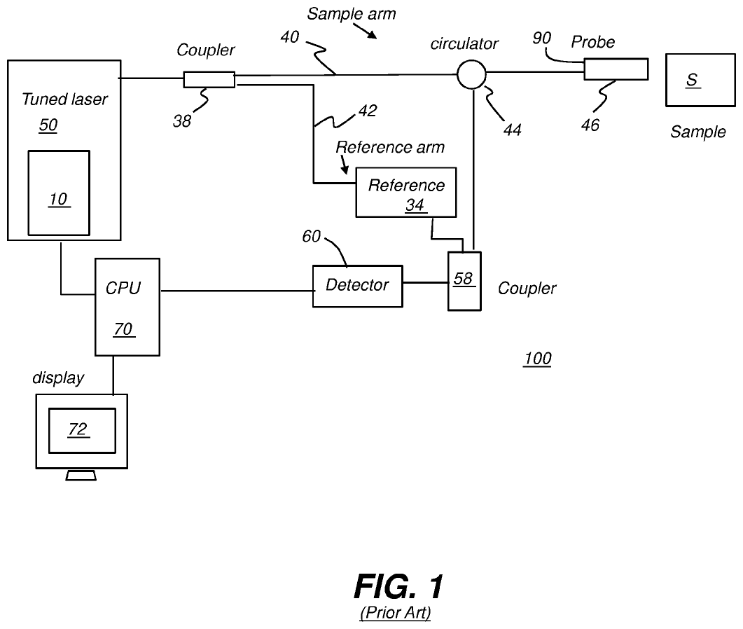

By way of example, the simplified schematic diagram of FIG. 1 shows the components of one type of OCT apparatus, here, a conventional swept-source OCT (SS-OCT) apparatus 100 using a Mach-Zehnder interferometer (MZI) system with a wavelength-tunable light source provided by a wavelength filter 10 that is part of a tuned laser source 50, which can be a laser, super-luminescent light-emitting diode (LED), super-continuum light source, or other type of wide-bandwidth light source. For intraoral OCT, for example, laser 50 can be tunable over a range of frequencies (expressed in terms of wave-numbers k) corresponding to wavelengths between about 400 and 1600 nm. According to an embodiment of the present disclosure, a tunable range of about 60 nm bandwidth centered about 1300 nm is used for intraoral OCT.

In the FIG. 1 device, the variable tuned laser 50 output goes through a coupler 38 and to a sample arm 40 and a reference arm 42. The sample arm 40 signal goes through a circulator 44 and is directed for imaging of a sample S from a handpiece or probe 46. The sampled signal is directed back through circulator 44 and to a detector 60 through a coupler 58. The reference arm 42 signal is directed by a reference 34, which can be a mirror or a light guide, through coupler 58 to detector 60. The detector 60 may use a pair of balanced photodetectors configured to cancel common mode noise.

Control logic processor (control processing unit CPU) 70 is in signal communication with tuned laser 50 and its programmable filter 10 and with detector 60. Processor 70 can control the scanning function of probe 46 and store any needed calibration data for obtaining a linear response to scan signals. Processor 70 obtains and processes the output from detector 60. CPU 70 is also in signal communication with a display 72 for command entry and OCT results display.

It should be noted that the swept-source architecture of FIG. 1 is one example configuration only; there are a number of ways in which the interferometer components could be arranged for providing swept-source OCT imaging.

Among proposed strategies for obtaining higher image acquisition speeds in an OCT system is simply using a high sweep-rate wavelength-tunable light source. However, as previously observed in the background section, the problem is more complex; attempts to operate at faster sweep rates have led to increased cost and can yield disappointing results with regards to the diagnostic benefits and overall quality of the OCT image content.

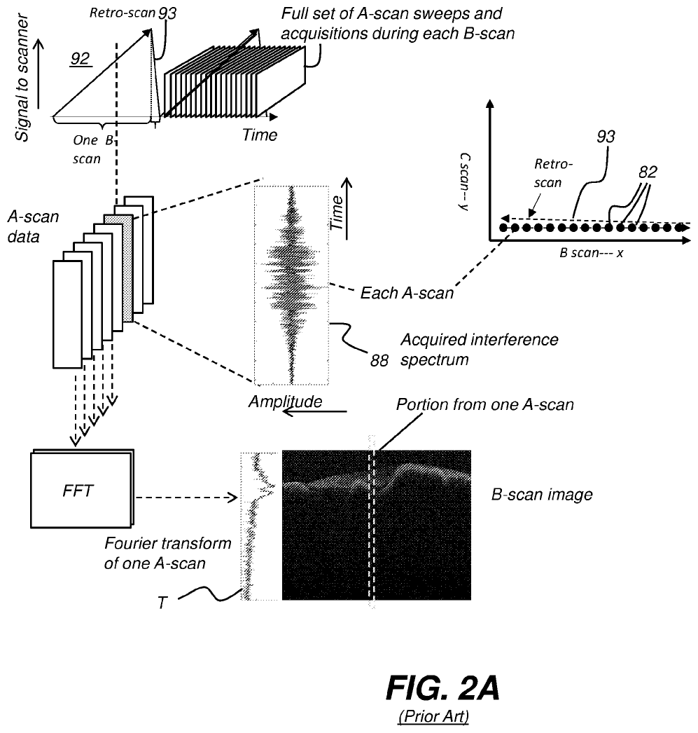

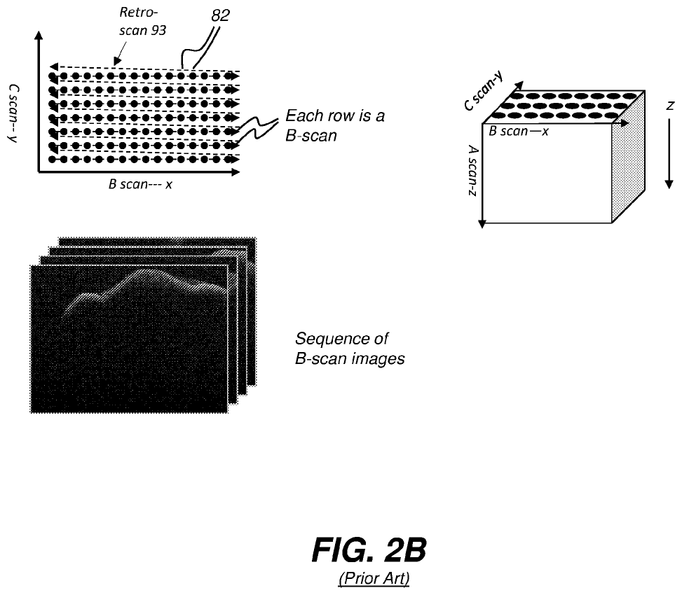

By way of further background, FIGS. 2A and 2B give an overview of the OCT scanning pattern as executed by probe 46. At each point in the scanning sequence, the OCT device performs an A-scan. A linear succession of A-scans then forms a B-scan, corresponding to the x-axis direction as shown. Successive B-scan rows, side-by-side, then form a C-scan which provides the 3D OCT image content for the sample S.

FIG. 2A schematically shows the information acquired during each A-scan. The scan signal for obtaining each B-scan image has two linear sections in the example shown, with a scan portion 92, during which the scanning mirror is driven to direct the sampling beam from a beginning to an ending position, and a retro-scan 93, during which the scanning mirror is restored to its beginning position. An interference signal 88, shown with DC signal content removed, is acquired over the time interval for each point 82, wherein the signal is a function of the time interval required for the sweep, with the signal that is acquired indicative of the spectral interference fringes generated by combining the light from reference and feedback sample arms of the interferometer (FIG. 1). The Fourier transform FFT generates a transform T for each A-scan. One transform signal corresponding to an A-scan is shown by way of example in FIG. 2A.

From the above description, it can be appreciated that a significant amount of data is acquired over a single B-scan sequence. In order to process this data efficiently, a Fast-Fourier Transform (FFT) is used, transforming the time-based signal data to corresponding frequency-based data from which image content can more readily be generated.

In Fourier domain OCT, the A scan corresponds to one line of spectrum acquisition which generates a line of depth (z-axis) resolved OCT signal. The B scan data generates a 2-D OCT image along the corresponding scanned line.

Raster scanning is used to obtain multiple B-scan data by incrementing the raster scanner 90 acquisition in the C-scan (y-axis) direction. This is represented schematically in FIG. 2B, which shows how 3-D volume information is generated using the A-, B-, and C-scan data.

The wavelength or frequency sweep sequence that is used at each A-scan point 82 can be modified from the ascending or descending wavelength sequence that is typically used. Arbitrary wavelength sequencing can alternately be used. In the case of arbitrary wavelength sequencing, which may be useful for some particular implementations of OCT, only a portion of the available wavelengths are provided as a result of each sweep. In arbitrary wavelength sequencing, each wavelength can be randomly selected, in arbitrary sequential order, to be used in the OCT system during a single sweep. A-scan points 82 can be uniformly spaced from each other with respect to the x axis, providing a substantially equal x-axis distance between adjacent points 82 along any B-scan image. Similarly, the distance between lines of scan points 82 for each B scan can be uniform with respect to the y axis. X-axis spacing may differ from y-axis spacing; alternately, spacing along these orthogonal axes of the scanned surface may be equal.

For conventional OCT approaches, image acquisition speed is related to factors of sweep rate and digitizer capability. Faster sweep rates can, in turn, allow improved A-scan frequencies, but at the cost of higher noise. High-speed digitization components are also needed at higher acquisition rates, with significant increase in component cost for the needed performance. Thus, there are some practical limits to scanning speed and overall OCT performance that can limit the use of OCT for chairside diagnosis and treatment.

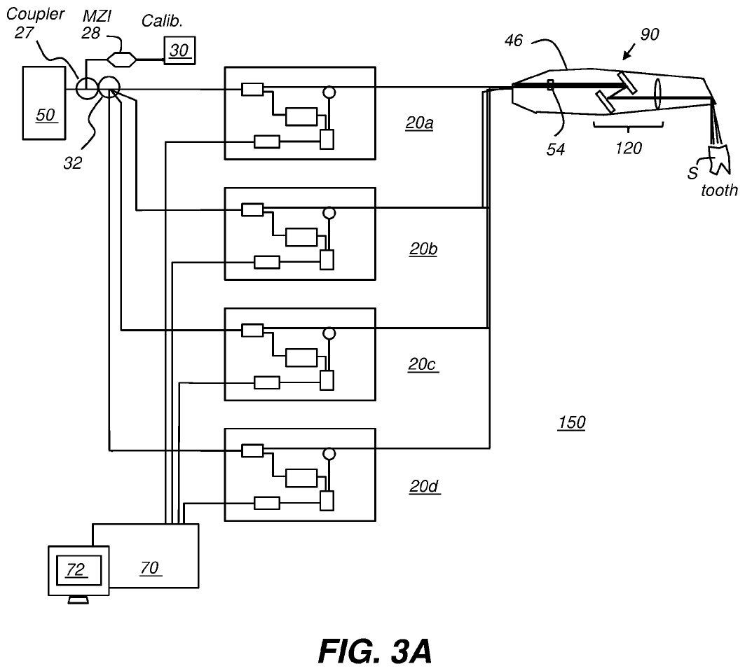

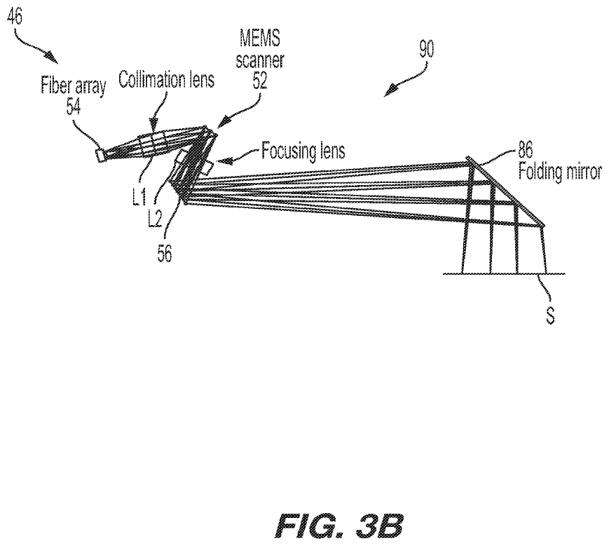

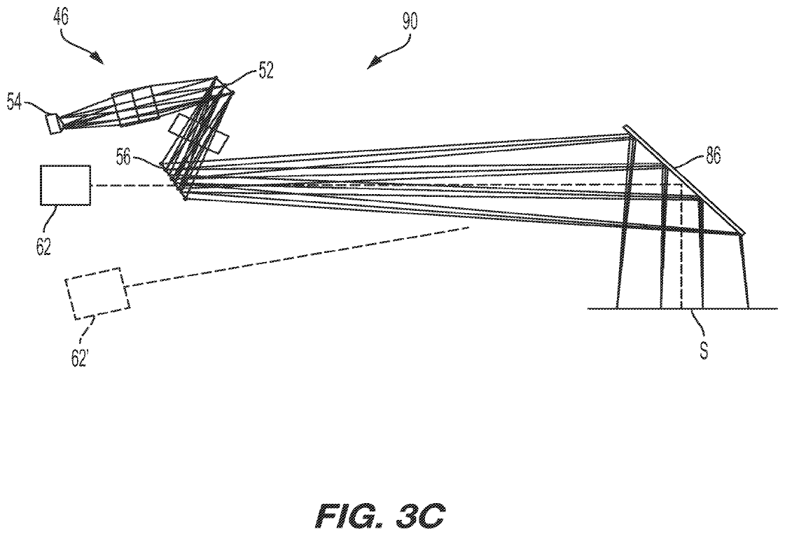

An embodiment of the present disclosure, shown schematically in FIG. 3A, addresses problems of image acquisition speed and the need for increased imaging range by using a multi-channel approach to dental OCT scanning and data acquisition. Referring to the schematic diagram of FIG. 3A, there is shown an exemplary high-speed intraoral OCT system 150 of the present disclosure having multiple channels that share a common scanning head 120, consisting of collimation lens. MEMS scanner, and focusing lens. For increasing amounts of scanning speed, the number of channels N can be two, three, or four, such as the four channels 20a, 20b, 20c, and 20d that lead to a scanning head 120 as shown in FIG. 3A. Additionally, five or more channels could be used, following the overall pattern described for four channels herein. The scanner 90 within probe 46 directs light originating from swept-wavelength laser source 50 in multiple channels to the tooth or other sample S.

As illustrated in FIG. 3A, a fiber coupler 27 splits off a small portion of the laser light to a Mach-Zehnder interferometer MZI 28. The interference light from the MZI is collected by a photodetector and additional circuit 30 to provide K-clock (K-trigger) signals, which are timing control triggers having equal wavenumber spacing defined in time. Given equal spacing of these signals, the OCT signal sampled with the K-clock timing is linear in wavenumber space. Alternately, the OCT signal can be resampled into a linear wavenumber space using the interference signal from MZI 28. (Zero crossing of the Mach-Zehnder interference (MZI) signal can be used to generate K-trigger signals to prompt the acquisition of SS-OCT signals.) The bulk of the swept-source laser 50 light output is fed into the multi-channel system for OCT imaging through a splitter 32, such as a PLC (Planar Lightwave Circuit) splitter. In each channel, the light illuminates a fiber optic interferometer that has a circulator 44 and a 90/10 fiber coupler 38 that splits light into reference and sample arms 42, 40 (FIG. 1). The system can optionally include additional detectors and optical components to provide polarization sensitive optical coherence tomography. Each channel directs light through probe 46 to a scanning head 120.

FIG. 3B shows probe 46 components that collimate, focus, and scan light from each of the four channels 20a, 20b, 20c, and 20d. As shown in the schematic of FIG. 3B, the multi-channel sampling arms are connected with a fiber array 54 inside of the scanner handpiece, probe 46, which can be used for intraoral or extraoral imaging. Connection of the variable wavelength light can be via a ribbon fiber (not shown). The fiber array 54 aligns the optical fiber cores precisely with desired pitch. The light from the fiber array goes through a collimation lens L1 and to a MEMS (micro-electromechanical systems) scanner 52 that is actuable to scan in one or two dimensions. Scanned light then goes through a focusing lens L2 as shown in FIG. 3B. This focused light reflects from a first folding mirror surface 56 and a second folding mirror surface 86 and is directed to sample S. Multiple spots are focused on the sample S surface with desired spacing; each spot is from one of the multiple channels 20a, 20b, 20c, and 20d.

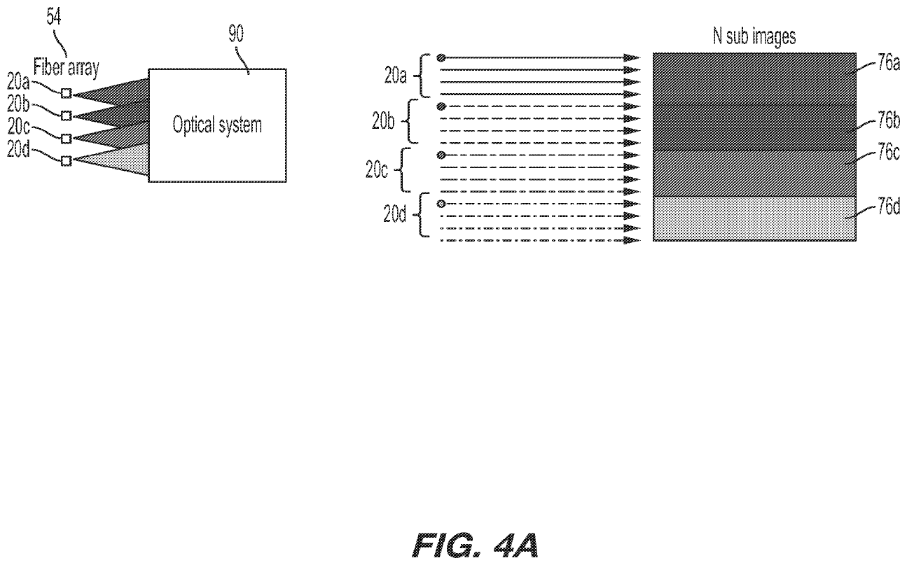

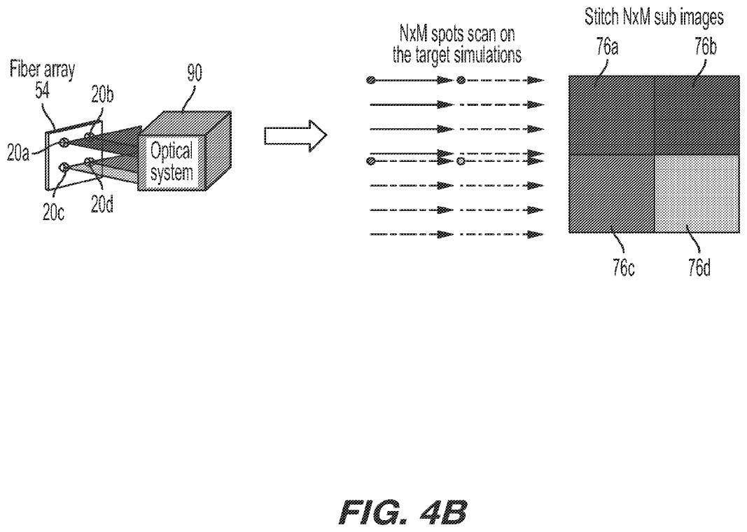

As is shown in the schematic of FIG. 3C, probe 46 can optionally include other components, such as a camera 62 for obtaining color information or to assist in probe movement, for example. Where camera 62 is used, surface 56 can be a dichroic surface, treated to reflect the IR light used for OCT scanning and to transmit visible light to the camera 62. A camera can alternately be provided at an oblique angle with respect to optical axis OA: by way of example, an alternate position of a camera 62′, which can be a second camera or the only camera, is shown in FIG. 4.

Fiber array 54 within probe 46 can have a number of different configurations. FIG. 4A shows fiber array 54 arranged in line as a one-dimensional (1D) array that simultaneously provides an output beam from each channel 20a, 20b, 20c, and 20d. The 1D array configuration can be used to direct the scanned beams to multiple spots, aligned on the target sample S. Scanning of a number N of illumination beams in this manner can be used to generate a number N of adjacent sub-images, shown as sub-images 76a, 76b, 76c, and 76d in the four-channel example of FIG. 4A. Processing software can then be used to stitch together the N adjacent images that lie along the scan line.

In scanning with a one-dimensional optical array using the FIG. 4A arrangement, the field of view (FOV) is divided in number of strips. Each focused spot from a channel scans only a small sub-region of the FOV. The reflected light from each focused spot at the sample is collected by probe 46 optics and is guided to the sampling arms of each channel. Light beams from the sample and reference arms 40 and 42 (FIG. 1) are recombined in the detection arms through a 50/50 coupler 58. Interference fringes that are formed are detected by balanced photo detectors or other mechanism in detector 60. The analogue signal from the balanced photo detector 60 can be digitized by a data acquisition card. The image volume from each channel can be generated using an OCT reconstruction algorithm. Finally, a reconstruction of the complete scanned image volume can be formed by stitching together the different sub-image volumes.

FIG. 4B shows an alternate arrangement using a 2×2 fiber array 54 to scan the FOV. This arrangement generates sub-image content as an array of images for stitching.

Since each channel scans only part of the field of view, the multi-channel system can achieve a much faster speed as compared to a single channel system. Using N multiple channels, scanning simultaneously, the complete FOV can be scanned in a fraction 1/N of the time required for the conventional single-channel arrangement.

Because the source laser output is split between N channels, some increase in laser power is needed in order to provide multi-channel OCT imaging capability. According to an embodiment of the present disclosure, a 40 mW laser is used to drive four channels, with output power subdivided to provide 10 mW in each channel.

In general, to achieve the same scanning speed, the swept laser source in an N-channel system only requires 1/N the sweep rate used in a single channel system. Lowering of the sweep rate accordingly lowers the digitization speed requirement of the data acquisition card, which can dramatically reduce the system cost.

To achieve the same imaging range, the frequency of the OCT signal, f_{OCT}, can be much lower with the multi-channel system than the frequency used in a single channel system. f_{OCT} may be expressed as follows:

$${f}_{OCT} = \frac{{f}_{s} Δ λ Z}{α {λ}^{2}} ,$$

wherein:
   Δλ is the bandwidth of the laser spectrum;
   λ is the central wavelength:
   Z is the imaging range;
   α is the duty cycle of the laser; and
   fₛ is the frequency of the swept laser source.

Since, in an N-channel system, the frequency of the OCT signal is only 1/N of the frequency used in a single channel system, the digitizer can operate at a lower sampling rate. Thus, N-channel design can reduce both cost and system noise. Alternatively, if the same high-speed digitizer that is used for a single scanner OCT probe is used in an N-channel system, performance can be improved, at up to N times of the imaging range.

Within the conventional interferometry system for each channel, the reference arm 42 typically includes some type of mirror or other reflective surface. The distance that light travels towards and back from the reflective surface, that is, the optical path delay for the reference arm, directly relates to a particular range within the sampled material. Thus, by adjusting the optical distance between the reflective or back-scattering material and interferometry combining components, returned light from variable depths within the sample contributes to the detection signal.

While conventional intraoral scanner embodiments can provide usable OCT image data following the design practices for single- and multi-channel devices described above, there is still considerable room for improvement. With respect to factors noted previously in the background section:
a. size, weight, and cost considerations become even more significant with multi-channel devices:
b. sensitivity to vibration and mechanical drift remain problems that are even more pronounced with multi-channel solutions; continued use or impact can have an adverse effect on instrument performance;
c. high maintenance and downtime costs can result where adjustments need to be performed related to optical path length differences between reference and sample arms;
d. manufacturing cost increases can result from the need to calibrate and package reference and sample arm components for multi-channel scanners;
e. insertion loss of each individual reference arm, compounded by problems such as dispersion mismatch, can require corresponding compensation or adjustment or can compromise image quality for one or more channels.

In response to the need for improved performance of the intraoral scanner with relation to accuracy, precision, and image quality, to help reduce component count, and to simplify or eliminate a number of problems particularly related to variability between channels, an embodiment of the present disclosure adapts a common path OCT design approach.

Unlike conventional Michelson interferometer design that uses separate sample and reference optical paths, common path OCT employs the same optical path for most of the sample and reference arms. Only the portion of the sample optical path between the probe and the imaged sample differs.

There have been various proposed configurations for using common path OCT such as in endoscopy and in dental imaging, including solutions that may combine sample and reference light paths, employing partial reflection from glass plate surfaces interposed between the sensing instrument and sample and using specialized optical systems using ball lenses, for example. Common-path configurations have been mentioned for supporting the use and adjustment of orthodontic aligners, for example. Conventional systems, however, have not provided suitable solutions using common path OCT techniques to support single-channel or multichannel intraoral scanning and to provide a compact hand-held intraoral probe using this approach and providing the needed usability, flexibility, and performance to support dental and related disciplines.

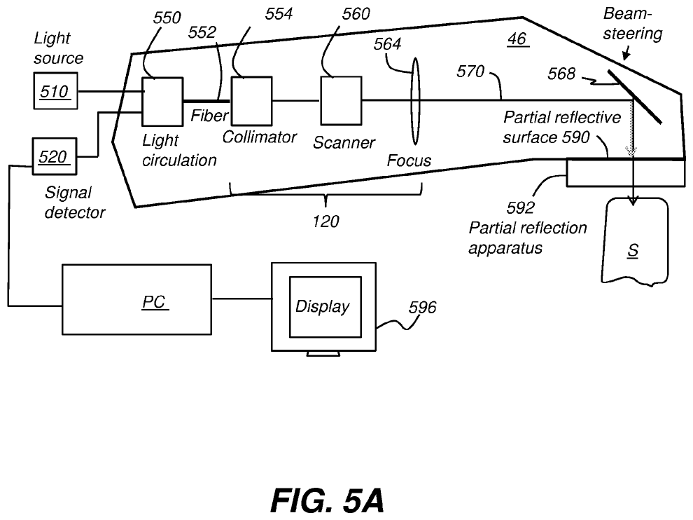

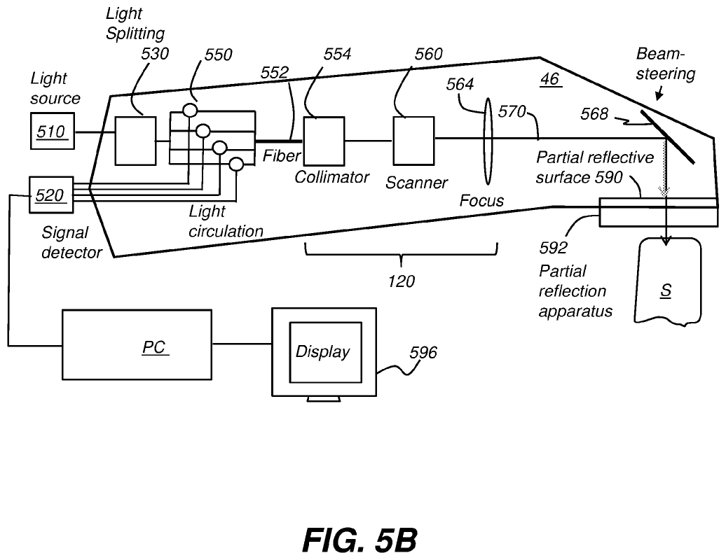

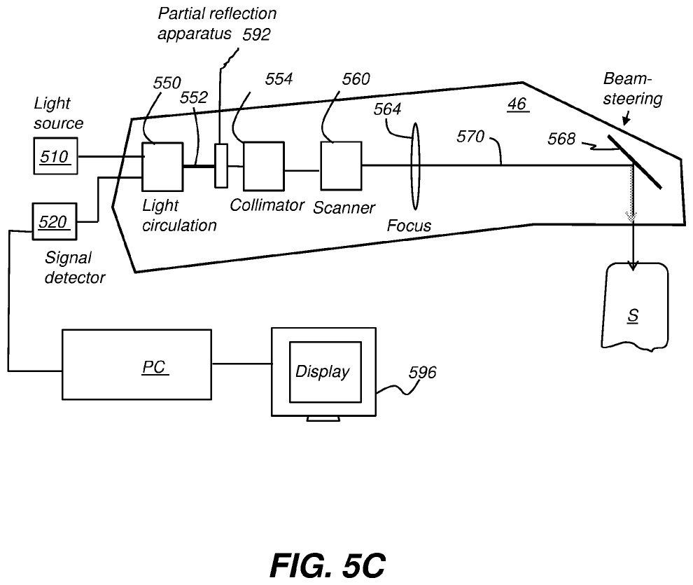

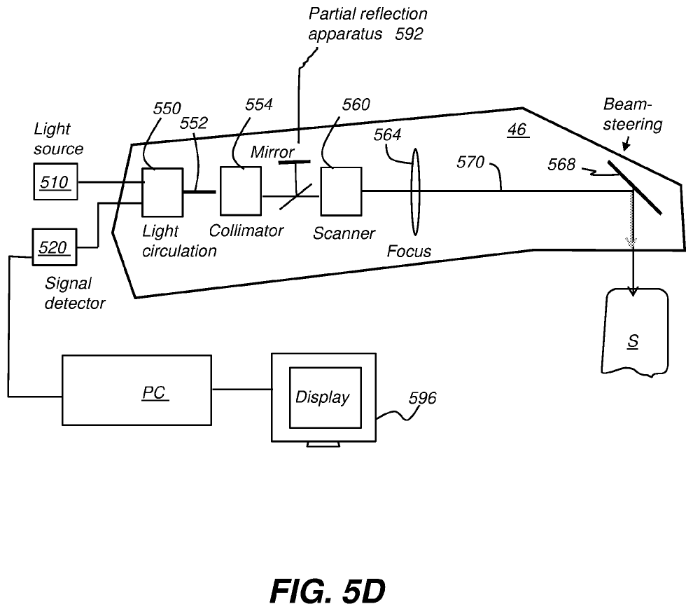

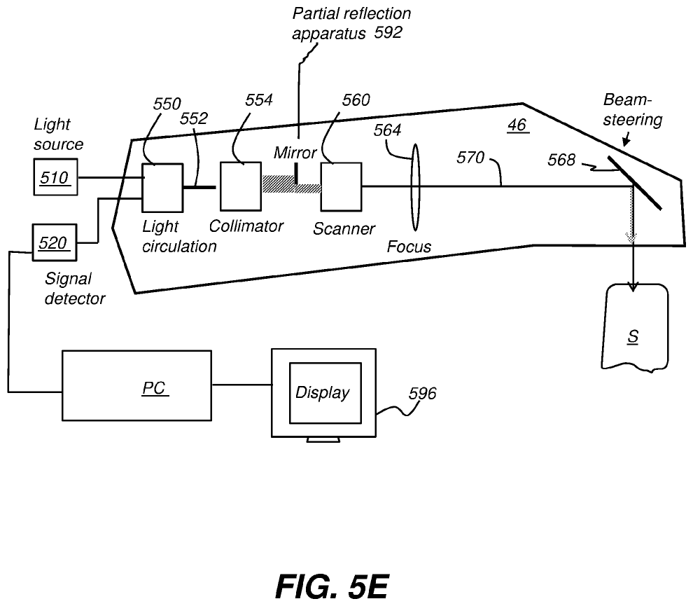

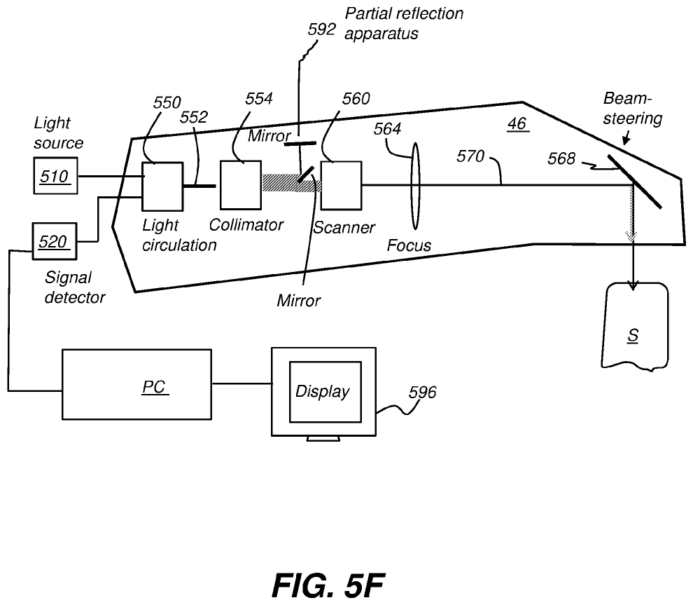

The schematic diagram of FIG. 5A shows components of probe 46 configured for common path OCT. A light source 510 provides wide-bandwidth source light to a light circulating subsystem 550. FIG. 5B expands the light circulating subsystem 550 in a multi-channel configuration. The light is initially split for individual channels through a beam splitting subsystem 530 before entering the corresponding circulating subsystems 550 for the multichannel configuration. Combined light for both sample and reference paths for each channel is conveyed by a fiber light guide 552. Fiber light guide 552 can be a single optical fiber for a single channel. For multiple channels, fiber light guide 552 can be an optical fiber bundle, having an optical fiber for each channel in the multi-channel imager. The light conveyed in light guide 552 goes to a collimator 554, which is a part of the scanning head 120. The collimator output is directed to a scanner 560 that scans the light through focusing optics 564 to a beam steering apparatus 568 that reflects the light 570 toward a partially reflective surface 590 near the output of probe 46, and then sample S. The partially reflective surface 590 can be part of a partial reflection apparatus 592, which can include a plate, window, optical wedge or other components, or the sample. The sample light path conveys light 570 through the partial reflection apparatus 592. The reference light path includes the reflected light from partially reflective surface 590. This light travels back through the optical system, along with scattered light returned from sample S. Light circulating subsystem 550 directs the signal light to a signal detection and processing apparatus 520 for generating the OCT signal having image content. If multiple channels are used, signal detection and processing apparatus 520) can be composed of multiple detectors, with each detector receiving a signal from its respective channel within circulating subsystem 550. A personal computer PC, such as a laptop or other portable computer or dedicated control logic processor apparatus can then provide the processed OCT image to a display 596.

Partial reflection apparatus 592 can employ a partially reflective surface such as a plate, a beam splitter with a mirror, one or more mirrors, or other at least partially reflective surface. The reflecting component can be positioned at any suitable position in the optical path, following the fiber light guide 552. FIGS. 5A and 5B show one embodiment in which the partial reflection apparatus is positioned at or near the output of probe 46. Another exemplary solution, shown in FIG. 5C, uses a partial reflection surface at the end of the light guide 552.

FIG. 5D shows the position of partial reflection apparatus 592 following the collimator, which includes, but is not limited to, a beam splitter and a back-reflecting mirror. The optical path length of the reference arm can be adjusted and fine-tuned by tuning the location of the mirror.

FIG. 5E shows a mirror as partial reflection apparatus 592: the mirror in this position clips and back-reflects part of the beam to provide reference signal (for illustrative purposes, the size of the beam in the figure is exaggerated.) The reference light power can be adjusted by translating the mirror across the optical path.

Alternately, the mirror can be tilted at an angle and the steered beam can be back-reflected by another mirror as shown in FIG. 5F. Both the power and optical path length of the reference light are adjustable. In addition, the sample beam can be spatially reshaped to improve the imaging quality or to serve a special need. For example, the central part of the beam can be steered as reference beam, forming a donut-shaped sample beam, which can provide large depth of field during imaging. The surface of the mirror can be flat, curved, or otherwise featured. The whole or the part of the mirror surface can be reflective. One or more parts of the surface can be transparent or semitransparent.

Partial reflective surface 590 can be formed using a coating or other suitable interface wherein the refractive index of the second medium is lower than the refractive index of the first medium.

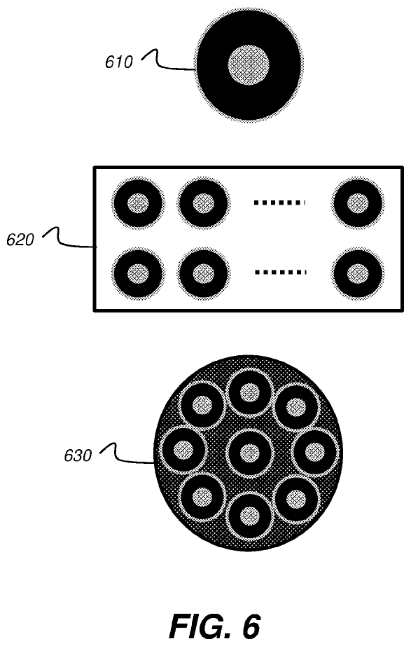

FIG. 6 shows various arrangements that can be used for fiber light guide 552 of FIG. 5. At top is shown a cross-section of a single fiber head 610. At center is shown a cross-section of a fiber array 620 having one or more rows and columns of fiber heads 610. At bottom is shown a fiber bundle 630.

In an alternate embodiment, light source 510 can be placed inside probe 46. In another alternate embodiment, signal detector 520 and associated detection and control electronics can also be placed inside probe 46.

The invention has been described in detail with particular reference to a presently understood exemplary embodiments, but it will be understood that variations and modifications can be effected within the spirit and scope of the disclosure.

For example, control logic processor 70 can be any of a number of types of logic processing device, including a computer or computer workstation, a dedicated host processor, a microprocessor, logic array, or other device that executes stored program logic instructions. The interferometer that is used for one or more channels, described in the example configurations given hereinabove as a type of Mach-Zehnder interferometer, can alternately be another appropriate type, such as a Michelson interferometer, for example, with appropriate component re-arrangement.

The presently disclosed exemplary embodiments are therefore considered in all respects to be illustrative and not restrictive. The scope of the disclosure is indicated by the appended claims, and all changes that come within the meaning and range of equivalents thereof are intended to be embraced therein.

Consistent with at least one exemplary embodiment, exemplary methods/apparatus can use a computer program with stored instructions that perform on image data that is accessed from an electronic memory. As can be appreciated by those skilled in the image processing arts, a computer program of an exemplary embodiment herein can be utilized by a suitable, general-purpose computer system, such as a personal computer or workstation. However, many other types of computer systems can be used to execute the computer program of described exemplary embodiments, including an arrangement of one or networked processors, for example.

A computer program for performing methods of certain exemplary embodiments described herein may be stored in a computer readable storage medium. This medium may comprise, for example; magnetic storage media such as a magnetic disk such as a hard drive or removable device or magnetic tape; optical storage media such as an optical disc, optical tape, or machine-readable optical encoding; solid state electronic storage devices such as random access memory (RAM), or read only memory (ROM); or any other physical device or medium employed to store a computer program. Computer programs for performing exemplary methods of described embodiments may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the application, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that can be directly associated with a display device and is periodically refreshed as needed in order to provide displayed data. This temporary storage buffer can also be considered to be a memory, as the term is used in the application. Memory is also used as the data workspace for executing and storing intermediate and final results of calculations and other processing. Computer-accessible memory can be volatile, non-volatile, or a hybrid combination of volatile and non-volatile types.

It will be understood that computer program products for exemplary embodiments herein may make use of various image manipulation algorithms and/or processes that are well known. It will be further understood that exemplary computer program product embodiments herein may embody algorithms and/or processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the application, are not specifically shown or described herein and may be selected from such algorithms, systems, hardware, components and elements known in the art.

Exemplary embodiments according to the application can include various features described herein (individually or in combination).

While the invention has been illustrated with respect to one or more implementations, alterations and/or modifications can be made to the illustrated examples without departing from the spirit and scope of the appended claims. In addition, while a particular feature of the invention can have been disclosed with respect to only one of several implementations/exemplary embodiments, such feature can be combined with one or more other features of the other implementations/exemplary embodiments as can be desired and advantageous for any given or particular function.

The term “a” or “at least one of” is used to mean one or more of the listed items can be selected. The term “about” indicates that the value listed can be somewhat altered, as long as the alteration does not result in nonconformance of the process or structure to the illustrated exemplary embodiment. Finally, “exemplary” indicates the description is used as an example, rather than implying that it is an ideal.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An optical coherence tomography scanner for imaging an intraoral sample, the scanner comprising:
a) a wavelength-tunable light source configured to generate scanning light having a range of wavelengths;
b) a scanning probe having a scanning head that directs light to the sample;
c) a light circulator configured:
(i) to direct the scanning light through at least a first optical fiber for conveying light to the scan head;
(ii) to direct a sample signal, having scattered and reflected light from the sample and through at least the first optical fiber, to a detector;
(iii) to direct a reference signal, having light reflected back from a partial reflection apparatus through at least the first optical fiber, to the detector;
d) wherein the detector forms a digital output signal indicative of interference of the combined sample and reference signals; and
e) a display configured to form an image of sample features according to the digital output signal.

2. The optical coherence tomography scanner of claim 1 wherein the partial reflection apparatus is disposed to reflect light from the at least the first optical fiber.

3. The optical coherence tomography scanner of claim 1 wherein the partial reflection apparatus is provided by a partially reflective surface of a window or a plate.

4. The optical coherence tomography scanner of claim 3 wherein the window or the plate is a wedge.

5. The optical coherence tomography scanner of claim 1 wherein the partial reflection apparatus consists of: (a) a beam splitter and a mirror, (b) a mirror that back reflects part of the light, or (c) a mirror that steers part of the light to a back-reflecting mirror.

6. (canceled)

7. (canceled)

8. The optical coherence tomography scanner of claim 5 wherein the partial reflection apparatus is the mirror that steers part of the light to the back-reflecting mirror and the part of the light that is steered is near the center.

9. The optical coherence tomography scanner of claim 1 wherein the scanning head is configured for intraoral scanning.

10. The optical coherence tomography scanner of claim 1 wherein the scanning head scans the light in one or two dimensions.

11. The optical coherence tomography scanner of claim 1 wherein at least one of: (a) the light circulator and first optical fiber are housed within the scanning probe, (b) the detector is housed within the scanning probe, or (c) the wavelength-tunable light source is housed within the scanning probe.

12. (canceled)

13. (canceled)

14. The optical coherence tomography scanner of claim 1 wherein the scanning probe is configured as a hand-held probe.

15. The optical coherence tomography scanner of claim 1 wherein the light circulator is configured to direct scanning light for a two or more channels.

16. An optical coherence tomography scanner for imaging an intraoral sample, the scanner comprising:
a) a wavelength-tunable light source configured to generate scanning light having a range of wavelengths;
b) a scanning probe having a scanning head that directs light to the sample;
c) a light circulator configured to direct light to and from a plurality of channels, each channel having:
(i) a signal detector;
(ii) an optical fiber in optical communication with the detector and the light source, for conveying the scanning light from the light source to the scanning head and for combining the scanning light that is reflected from a partial reflection apparatus with scattered and reflected light from the sample;
d) wherein the detector forms a digital output signal indicative of interference of the combined light from each of the plurality of channels; and
e) a display configured to form an image of sample features according to the digital output signal.

17. The optical coherence tomography scanner of claim 16 wherein the partial reflection apparatus receives light from the optical fiber.

18. The optical coherence tomography scanner of claim 16 wherein the partial reflection apparatus is provided by a partially reflective surface of a window or a plate.

19. The optical coherence tomography scanner of claim 18 wherein the window or the plate is a wedge.

20. The optical coherence tomography scanner of claim 16 wherein the partial reflection apparatus consists of: (a) a beam splitter and a mirror, (b) a mirror that back-reflects part of the light or (c) a mirror that steers part of the light to a back-reflecting mirror.

21. (canceled)

22. (canceled)

23. The optical coherence tomography scanner of claim 20 wherein the partial reflection apparatus is the mirror that steers part of the light to a back-reflecting mirror and the part of the light that is steered is near the center of the mirror.

24. The optical coherence tomography scanner of claim 16 wherein the light circulator is housed together with the scanning head inside the scanning probe.

25. The optical coherence tomography scanner of claim 16 wherein the scanning head scans the light in one or two dimensions.

26. The optical coherence tomography scanner of claim 16 wherein the scanning probe is a hand-held probe.

27. The optical coherence tomography scanner of claim 16 wherein the scanning head is configured for intraoral scanning.
